(19) United States
(12) Patent Application Publication
(10) Pub. No.: US 2006/0189579 A1
(43) Pub. Date: Aug. 24, 2006

(54) Method of stabilizing ascorbyl phosphate and salts thereof

(76) Inventors: Stewart Anderson, Munchwilen (CH); Martin Gadient, Basel (CH); Peter Hofmann, Weil-Haltingen (DE); Jonathan W. Wilson, Ringwood, NJ (US)

Correspondence Address: NIXON & VANDERHYE, PC, 901 NORTH GLEBE ROAD, 11TH FLOOR, ARLINGTON, VA, 22203, US

(21) Appl. No.: 10/564,443

(22) PCT Filed: Sep. 2, 2004
(86) PCT No.: PCT/EP04/09781

§ 371 Date: Jan. 12, 2006

(30) Foreign Application Priority Data
Sep. 2, 2003 (EP) 03019881.6

(51) Int. Cl. A61K 31/665; A61K 9/50

(52) U.S. Cl. 514/100; 424/490

(57) ABSTRACT

Ascorbyl phosphate is stabilized against degradation by phosphatases by coating with a lipid.

The present invention relates to a method of stabilizing ascorbyl phosphates against degradation by phosphatases. Ascorbyl phosphates are used in feedstuff industry as an additive to feed for pets and other animals. Such feed may contain active phosphatases which may lead to degradation of ascorbyl phosphates. It has now been found that ascorbyl phosphates can be stabilized against degradation by phosphatases by coating with lipids. Thus, the present invention relates to a method of stabilizing ascorbyl phosphates against degradation by phosphatases by coating with lipids. The invention further relates to novel compositions comprising certain lipid-coated ascorbyl phosphates as well as animal feed and feed premixes containing them.

Vitamin C preparations which are coated with lipids to stabilize vitamin C against the impact of external influences such as atmosphere, moisture, light and heat are known from EP 0 443 743. These known preparations mandatorily contain vitamin E in the coating. By the present invention, it has surprisingly been found that ascorbyl phosphates can be stabilized against phosphatases by lipid coating in the absence of vitamin E. Indicating that primary stressor for phosphorylated vitamin C is not oxidation.

The term “ascorbyl phosphate” as used herein denotes metal salts of mono- and poly-phosphoric acid esters of ascorbic acid wherein the phosphorylated hydroxy group of the ascorbic acid molecule features one or more phosphoric acid (phosphate) units, and metal cations, e.g. sodium and/or calcium ions, are also present. “poly” generally denotes 2-10, preferably 2-4, phosphate units. The “ascorbyl phosphates” may also be referred to in general as “ascorbyl (poly)phosphates” to embrace both mono- and polyphosphates.

Typical ascorbyl phosphates for use in the present invention are trisodium L-ascorbyl-2-monophosphate and sodium calcium L-ascorbyl-2-polyphosphate (principally triphosphate) which are commercially available as STAY-C50 and ROVIMIX STAY-C35, respectively (Roche Vitamins AG, Basel). The amount of ascorbyl phosphate or salt thereof in the stabilized compositions is such to provide between about 5 wt.-% to about 40 wt.-%, based on ascorbic acid equivalents.

The term “lipid” as used herein comprises mono-, di- and triglycerides of fatty acids, fatty acid sucrose and propyleneglycol esters and the like, and waxes, phospholipids and sugar lipids, as well as mixtures of the foregoing. Particular examples of lipids are soybean oil, palm oil, rapeseed oil, coconut oil, and the like. Preferred are hydrogenated plant oils and glycerol stearates. The amount of lipid is about 10 wt.-% to about 60 wt.-%, based on the total weight of the composition.

The coated ascorbyl phosphate compositions may further contain adsorbants, e.g., polysaccharides such as starch and modified starch, or calcium silicate alone or a mixture of calcium silicate with one of the following mixture components: microcrystalline cellulose, magnesium silicate, magnesium oxide, stearic acid, calcium stearate, magnesium stearate, hydrophilic silicic acid, dicalcium phosphate, tricalcium phosphate and kaolin. The amount of adsorbant, if present, is about 0.5 wt.-% to about 5 wt.-%, based on the total weight of the composition.

The coated ascorbyl phosphate compositions can be manufactured by dispersing the ascorbyl phosphate in the liquified (by heating) lipid and subsequent processing the melt into a solid composition, e.g., a granulate by procedures known per se, e.g. by spraying the melt in cold air, or by coating the ascorbyl phosphate with liquid lipid in a fluidized bed.

The so obtained granulate is, suitably, collected in or coated by an adsorbant, e.g., such as disclosed above. The so-obtained granulate is stabilized against degradation by phosphatases. When 10 mg of granulate containing 15% of ascorbic acid activity were exposed during 60 minutes to 30 mg of acid phosphatase (Roche Diagnostics GmbH, Mannheim, Germany) diluted in 20 ml of water, more than 80% of the ascorbyl phosphate remained unchanged. Moreover, the granulate is free-flowing, non-dusting and non-caking.

Granulate compositions as disclosed above, wherein the ascorbyl phosphate is trisodium L-ascorbyl-2-monophosphate, or sodium calcium L-ascorbyl-2-polyphosphate, and mixtures thereof, are novel and, as such, also are an object of the present invention.

The coated ascorbyl phosphate composition is conventionally mixed into a premix containing in addition vitamin, minerals and other additives. The premix is added to feed, mixed, stored and then subjected to a hydrothermal treatment, e.g. pelleting, extrusion or retorting to produce diets for animals such as pets, productive livestock and fish. The diets typically contain components of animal origin, such as fish, fish parts, fish meal, fats, meat, meat byproducts. Accordingly, it is important that the incorporated composition protects the ascorbic acid activity from extended exposure to phosphatases present in these components. The preparation also protects the ascorbic acid activity from degradation by phosphatases present in the digestive tract of animals. For instance, it reduces decomposition of ascorbyl phosphate in the stomach of ruminants and thus provides larger amounts of vitamin C to its site of absorption.

The following Examples illustrate the invention further.

### EXAMPLE 1

300 g of hardened palm oil having a melting point of 46° C. were heated to 75° C. 400 g of ROVIMIX STAY-C 35, which contain 140 g ascorbic acid activity, were added to a fluidized air bed granulator. The oil was then added to the fluidized bed and the resulting powder collected.

There was obtained a beige, granulated powder with a content of 20% ascorbic acid activity. When 10 g of the product was exposed to humid air of 75% rH, weight increased was 0.27 g and the product remained free-flowing.

### EXAMPLE 2

112 g of glycerol monostearate having a melting point of 60° C. were heated to 90° C. 888 g of STAY-C 50, which contain 400 g ascorbic activity, were added to a fluidized air bed granulator. The oil was then added to the fluidized bed and the resulting powder collected. 10 g of hydrophobic silicic acid were then added. The resulting powder contained 40% ascorbic acid activity. The product passed through an Agway flow tunnel having an opening of 11 mm.

### EXAMPLE 3

1346 g of of castor oil having a melting point of 65° C. were heated to 90° C. 1000 g of ROVIMIX STAY-C 35, which contained 350 g ascorbic activity, were slowly added to the vessel and dispersed in the oil. The mixture was then sprayed in a fluidized bed flushed with cool air of 5° C. The resulting powder was collected. The resulting powder was free-flowing and contained 15% ascorbic acid activity.

1. A method of stabilizing an ascorbyl (poly) phosphate against degradation by phosphatases which comprises coating said ascorbyl (poly)phosphate with a lipid.

2. A method as in claim 1 wherein the ascorbyl (poly)phosphate salt is trisodium ascorbyl-2-monophosphate, or sodium calcium ascorbyl-2-polyphosphate.

3. A method as in claim 1 wherein the lipid is a wax or plant oil.

4. A method as in claim 1 wherein an adsorbant is additionally present.

5. A method as in claim 1 the amount of ascorbyl (poly) phosphate is such to provide between about wt. 5% to about 40 wt.-% ascorbic acid equivalents.

6. A method as in claim 1 wherein the amount of lipid is about 10 wt.-% to about 60 wt.-%, based on the total weight of the composition.

7. A method as in claim 1 wherein an adsorbant is present in an amount of about 0.5 wt.-% to about 5 wt.-%, based on the total weight of the composition.

8. A method as in claim 1 wherein a granulate having an average mean particle size of about 0.1 to about 1.0 mm is prepared.

9. Granulate compositions comprising trisodium L-ascorbyl-2-monophosphate or sodium calcium L-ascorbyl-2-polyphosphate or mixtures thereof, and a lipid.

10. Granulate compositions as in claim 9 wherein the lipid is a wax or plant oil.

11. Granulate compositions as in claim 9 wherein the amount of trisodium L-ascorbyl-2-monophosphate or sodium calcium L-ascorbyl-2-polyphosphate in the granulate is such to provide between about 5-% to about 40 wt.-%, ascorbic acid equivalents.

12. Granulate compositions as in claim 9 wherein the amount of lipid is about 10 wt.-% to about 60 wt.-%, based on the total weight of the composition.

13. Granulate compositions as in claim 9 wherein the amount of adsorbant, if present, is about 0.5 wt.-% to about 5 wt.-%, based on the total weight of the composition.

14. Granulate compositions as in claim 9 wherein the granulate has a mean particle size of about 0.1 to about 1.0 mm.

15. Animal feed and feed premixes containing a granulate composition as claimed in claim 9.

16. Animal feed and feed premixes as in claim 15 for ruminants.
